(19) United States
(12) Patent Application Publication
(10) Pub. No.: US 2018/0271416 A1
(43) Pub. Date: Sep. 27, 2018

(54) HEADGEAR-MOUNTED SWEAT SENSING DEVICES

(71) Applicant: Eccrine Systems, Inc., Cincinnati, OH (US)

(72) Inventors: Gavi Begtrup, Cincinnati, OH (US); Robert Beech, Cincinnati, OH (US); Austin Morgan, Bellevue, KY (US)

(73) Assignee: Eccrine Systems, Inc., Cincinnati, OH (US)

(21) Appl. No.: 15/762,592

(22) PCT Filed: Sep. 25, 2016
(86) PCT No.: PCT/US16/53625

§ 371 Date: Mar. 23, 2018

Related U.S. Application Data
(60) Provisional application No. 62/232,799, filed on Sep. 25, 2015.

(51) Int. Cl. A61B 5/145; A61B 10/00; A61B 5/00

(52) U.S. Cl. CPC A61B 5/14517; A61B 10/0064; A61B 5/6814; A61B 5/6803; A61B 5/4266

(57) ABSTRACT

The disclosed invention incorporates sweat sensing devices into headgear so that accurate biofluid analyte measurements may be made during physical activity. As disclosed herein, a sweat sensing device may be incorporated into an inner surface or support structure of headgear, including hardhats, sports headgear, flight helmets, combat helmets, sweatbands, sports caps, visors, and masks. The device is further configured to recognize and alter operational states when the device is not in adequate skin contact for operation. Some embodiments are fully disposable, and other embodiments include a reusable component that may be integrated into, or attached to, the headgear.

### CROSS-REFERENCE TO RELATED APPLICATIONS

The present application claims priority to U.S. Provisional Application No. 62/232,799, filed Sep. 25, 2015; and PCT/US16/53625, filed Sep. 25, 2016; and has specification that builds upon PCT/US16/43771, filed Jul. 23, 2016; and PCT/US2016/59392, filed Oct. 28, 2016, the disclosures of which are hereby incorporated herein by reference in their entirety.

### BACKGROUND OF THE INVENTION

Sweat sensing technologies have enormous potential for applications ranging from athletics, to neonatology, to pharmacological monitoring, to personal digital health, to name a few applications. This is because sweat contains many of the same biomarkers, chemicals, or solutes that are carried in blood, which can provide significant information which enables one to diagnose ailments, health status, toxins, performance, and other physiological attributes even in advance of any physical sign. Furthermore, sweat itself, and the action of sweating, or other parameters, attributes, solutes, or features on or near skin or beneath the skin, can be measured to further reveal physiological information.

Of all the other physiological fluids used for bio monitoring (e.g., blood, urine, saliva, tears), sweat has arguably the least predictable sampling rate in the absence of technological solutions. An excellent summary is provided by Sonner, et al. in the 2015 article titled “The microfluidics of the eccrine sweat gland, including biomarker partitioning, transport, and biosensing implications,” Biomicrofluidics 9, 031301, herein included by reference. However, with proper application of technology, sweat can be made to outperform other non-invasive or less invasive biofluids in predictable sampling. Many of the drawbacks and limitations of the sweat medium can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sweat sensing and stimulating technology into intimate proximity with sweat as it is generated. With the improvements embodied in the current invention, sweat sensing can become a compelling new biosensing medium.

In particular, sweat sensing devices hold tremendous promise for use in workplace safety, athletic, military, and clinical diagnostic settings. For many of these applications to be effective, however, it is desirable that the patch be comfortably integrated into headgear equipment, such as a helmet or hardhat, while maintaining adequate contact with the skin. As disclosed herein, a sweat sensing device is incorporated into an inner surface or support structure of headgear for use in physically active conditions.

### Definitions

Before continuing with the background, a variety of definitions should be made, these definitions gaining further appreciation and scope in the detailed description and embodiments of the disclosed invention.

“Sweat sensor” means any type of sensor that measures a state, presence, flow rate, solute concentration, solute presence, in absolute, relative, trending, or other ways in biofluid. Sweat sensors can include, for example, potentiometric, amperometric, impedance, optical, mechanical, antibody, peptide, aptamer, or other means known by those skilled in the art of sensing or biosensing.

“Analyte” means a substance, molecule, ion, or other material that is measured by a sweat sensing device.

“Measured” can imply an exact or precise quantitative measurement and can include broader meanings such as, for example, measuring a relative amount of change of something. Measured can also imply a binary measurement, such as ‘yes’ or ‘no’ type measurements.

As used herein, “biofluid” is a fluid that is comprised mainly of interstitial fluid or sweat as it emerges from the skin. For example, a fluid that is 45% interstitial fluid, 45% sweat, and 10% blood is a biofluid as used herein. For example, a fluid that is 20% interstitial fluid, 20% sweat, and 60% blood is not a biofluid as used herein. For example, a fluid that is 100% sweat or 100% interstitial fluid is a biofluid. A biofluid may be diluted with water or other solvents inside a device because the term biofluid refers to the state of the fluid as it emerges from the skin.

“Chronological assurance” means the sampling rate or sampling interval that assures measurement(s) of analytes in biofluid in terms of the rate at which measurements can be made of new biofluid analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5 to 30 minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

As used herein, the term “analyte-specific sensor” is a sensor specific to an analyte and performs specific chemical recognition of the analytes presence or concentration (e.g., ion-selective electrodes, enzymatic sensors, electro-chemical aptamer based sensors, etc.). For example, sensors that sense impedance or conductance of a fluid, such as biofluid, are excluded from the definition of “analyte-specific sensor” because sensing impedance or conductance merges measurements of all ions in biofluid (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

“Sweat sensor data” means all of the information collected by device sensor(s) and communicated via the device to a user or a data aggregation location.

“Correlated aggregated sweat sensor data” means sweat sensor data that has been collected in a data aggregation location and correlated with relevant outside information such as time, temperature, weather, location, user profile, other sweat sensor data, or any other relevant data.

“EAB sensor” means an electronic aptamer-based sensor, such as is disclosed in U.S. Pat. Nos. 7,803,542 and 8,003,374.

“Operation and compliance warning” means an alert generated by the sweat sensing device and relayed to the system user if a reading indicates a device is not in adequate skin contact.

This has served as a background for the disclosed invention, including background technical invention needed to fully appreciate the disclosed invention, which will now be summarized.

### SUMMARY OF THE INVENTION

The disclosed invention addresses a difficulty involving the use of sweat sensing devices as part of a biological monitoring system by incorporating a sweat sensing device into headgear, while maintaining skin contact that is calibrated to allow accurate sweat analyte measurements.

### BRIEF DESCRIPTION OF THE DRAWINGS

The objects and advantages of the disclosure will be further appreciated in light of the following detailed descriptions and drawings in which:

FIG. 1A is a representation of at least a portion of the disclosed invention including a mechanism for incorporating a fully disposable sweat sensing device into headgear.

FIG. 1B is a top down view of at least a portion of the disclosed invention including a mechanism for incorporating a fully disposable sweat sensing device into headgear.

FIG. 2 is an example embodiment of at least a portion of a device of the disclosed invention including a mechanism for incorporating a reusable sweat sensing device component into headgear.

FIG. 3 is an example embodiment of at least a portion of the disclosed invention including a mechanism for incorporating a sweat sensing device into headgear, where the device has a reusable component and a disposable component.

FIG. 4 is an example embodiment of at least a portion of a device of the disclosed invention including a reusable component and a disposable component that are incorporated into headgear.

FIG. 5 is an example embodiment of at least a portion of a device of the disclosed invention including a reusable component and a disposable component that are incorporated into headgear.

FIG. 6 is an example embodiment of at least a portion of a device of the disclosed invention including a reusable component and a disposable component that are incorporated into headgear.

FIG. 7 is a representation of at least a portion of a device of the disclosed invention, where the device has a protective film.

### DETAILED DESCRIPTION OF THE INVENTION

The present disclosure provides a device capable of being incorporated into headgear systems that provides sweat sensor data capable of translation into physiological information about the wearer to enhance safety and improve performance.

Physiologically, the forehead area is an ideal location for collecting data with a sweat sensing device. Compared to other locations on the body, the eccrine sweat glands of the forehead readily produce sweat, even at lower sweat threshold temperatures. This allows multiple or near-continuous sweat measurements to reliably take place with minimal sweat stimulation. The skin surface of the forehead is also relatively smooth, lacks substantial hair, and benefits from the support of underlying bone structure. These features facilitate close fluidic contact between sweat sensors and newly emerging sweat, thereby reducing risk of contamination by surface contaminants and old sweat. Additionally, these features help reduce sweat volumes beneath the sensor, which allows the device to take measurements at lower sweat generation rates, allowing relatively more chronologically assured measurements per unit time, and enhancing detection of large, slow-diffusing analytes.

The forehead location is also advantageous because of the widespread use of headgear for multiple applications. Such headgear, such as military flight helmets, industrial hardhats and visors, and sports helmets, are increasingly outfitted with communications and sensing devices to improve the safety and performance of the wearer. Locating a sweat sensing device in headgear, therefore allows the device to use existing communications, processing and power infrastructures, and adds the capability of measuring and interpreting biomarkers in real time as they emerge from the wearer. Other headgear formats, such as sweat bands, caps, visors, and watch caps, will also benefit from incorporated sweat sensing devices, as electronics continue to miniaturize.

However, the incorporation of sweat sensing devices into headgear also presents several challenges, including the potential for electrical interference or abrasion to device sensors caused by contact with the forehead or skin if the sensors are not properly shielded or otherwise protected. Further, use with headgear may result in sensor output variations caused by the motion of the wearer's head, relative motion between the wearer's head and the headgear, and pressure variations between the headgear, the device, and the wearer's skin.

The present disclosure applies at least to any type of sweat sensing device that measures sweat, biofluid, sweat generation rate, sweat chronological assurance, its solutes, solutes that transfer into sweat from skin, a property of or things on the surface of skin, or properties or things beneath the skin. The disclosure applies to sweat sensing devices which can take on forms including patches, bands, straps, portions of clothing or equipment, or any suitable mechanism that reliably brings sweat stimulating, sweat collecting, and/or sweat sensing technology into intimate proximity with biofluid as it is generated.

Certain embodiments of the invention show sensors as simple individual elements. It is understood that many sensors require two or more electrodes, reference electrodes, or additional supporting technology or features that are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referenced herein by what the sensor is sensing, for example: an analyte-specific sensor; an impedance sensor; a sweat volume sensor; a sweat generation rate sensor; and a solute generation rate sensor. Certain embodiments of the disclosed invention show sub-components of what would be sweat sensing devices with more sub-components needed for use of the device in various applications, which are obvious (such as a battery), and for purpose of brevity and focus on inventive aspects, are not explicitly shown in the diagrams or described in the embodiments of the present disclosure.

Use of a sweat sensing device within headgear as disclosed presents a potentially difficult environment for proper sweat sensor function. As is discussed in PCT/US16/43771, when ionophore sweat sensors are placed directly in contact with skin, they can be subject to failure due to the delamination of ionophore membranes from the sensor. This is a particularly acute problem for sweat sensing devices mounted in helmets or other headgear, which are worn for long periods of time, usually in physically active applications that subject the device to a great deal of movement relative to the wearer. As discussed, comfortable wear of such devices requires a somewhat flexible interface between the sweat sensing devices and skin, however, such an interface would increase abrasion and sensor failure for sensors placed directly against the skin. Further, as disclosed in PCT/US2016/59392, electrical noise from the body can also interfere with sweat analyte measurements if analyte-specific sensors are placed in direct contact with skin. Therefore, in certain embodiments of the disclosed invention, sweat sensors will be configured to remain out of direct physical contact with the wearer's skin. In such embodiments, sweat may be wicked off the skin and across the analyte-specific sensors for analyte detection. Alternatively, sweat sensors may be separated from skin by a layer of protective material deposited on the sensors. In some embodiments, the devices may also have electromagnetic shielding materials between the sensors and skin.

The invention also includes a means to determine if the sweat sensing device is being worn by an individual, and whether it is in proper skin contact to allow accurate sweat sensing device readings, as disclosed in PCT/US15/55756, which is incorporated herein in its entirety. This may be accomplished through skin impedance electrodes or by use of capacitive sensor electrodes, as are commonly used in consumer wearable health monitoring devices and mobile computing devices. If impedance electrode contact with the skin is, or becomes inadequate, this can be detected as an increase in impedance and the device can send an alert signal to the user or another device. Similarly, capacitance sensors may be placed on selected locations on the skin-facing side of the device, and could convey information about the distance between the device and the skin. Inadequate contact can indicate that the device has been removed by the user, or has become detached from the skin for other reasons.

Also during use, the sweat sensing device's skin contact sensor may continuously or near-continuously monitor the adequacy of skin contact. During times of poor or no skin contact, the device may avoid taking measurements, or may, via algorithm, account for the poor or no skin contact when weighting the measurements. The device may also communicate to the wearer or user to inform them of the inadequacy or absence of skin contact and to advise corrective action. Alternately, the device may track periods during which the device is out of contact with skin (when the headgear is removed) and discard any collected data, or extrapolate previous measurements to bridge gaps in device use.

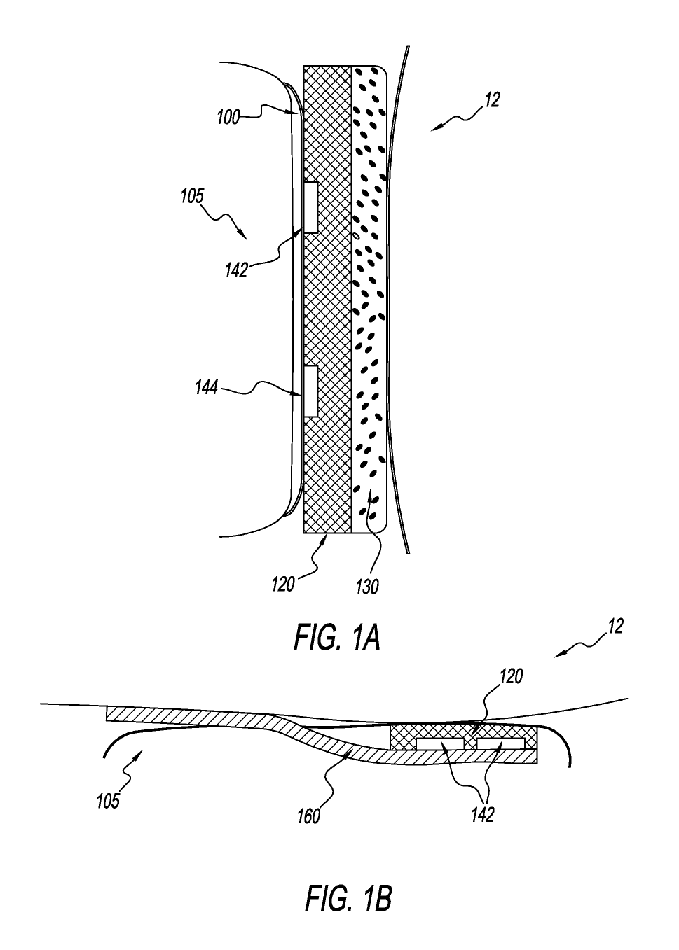

With reference to FIG. 1A, in an example embodiment of the disclosed device having a fully disposable configuration, adhesive layer 100 secures the disposable sweat sensing device component 120 to the mounting surface of headgear 105 so that when the headgear is worn by an individual, the device 120 will be in contact with the wearer's skin 12, for example, at the wearer's forehead. In addition to remaining in contact with the skin 12, the device should be held against the skin with positive and consistent pressure that is great enough to allow accurate device operation, but not so great as to impair operation. For example, when using ion selective electrode (ISE) sensors for detecting analytes like Na⁺, Cl⁻, and K⁺, the minimum pressure for proper operation of an embodiment of the disclosed invention was about 265 pascals, and the maximum pressure the device could endure before ISE failure was around 75,000 pascals. Pressures below the minimum amount would not maintain adequate contact between the ISE sensors and sweat samples, and higher pressures caused the ionophore coatings to delaminate and fail. Fluctuations in pressure can also increase electrical noise, and cause ionophore delamination, therefore consistent pressure is also required in order to allow proper operation. Other sensor modalities, such as EAB sensors, may be similarly sensitive to pressure and pressure changes.

The mounting surface 105 may be, for example, a suspension-type support structure for a hardhat, the forehead pad structure of a sports helmet, a military flight helmet, or other helmet. Alternatively, the mounting surface 105 may be a flexible or semi-flexible headband, or any other device that can comfortably secure the sweat sensing device next to the skin. Other suitable attachment means may be used as long as consistent pressure within the specified range is maintained during use. Adequate and consistent pressure may be maintained through use of a spacer component, such as a spring, sponge, or foam block that presses the device against the skin in the necessary pressure range. In other embodiments, the pressure provided by the mounting surface will be calibrated to facilitate proper device operation, for example through a strap adjustment mechanism.

With further reference to FIG. 1A, the device is flexibly secured to the wearer's skin 12 by use of a skin interface layer 130. The interface layer 130 may be made of any suitable material that creates a flexible bond between the device and the wearer's skin that allows the headgear to move comfortably during active use, but keeps the device relatively stationary and in contact with the wearer's skin. The interface layer 130 may be from 6 μm to several mm thick. Preferably, the interface layer 130 will be thin, e.g., 15 μm, to minimize dead volume between the skin and the device, thereby improving the device's chronologically assured resolution. The interface layer 130 may be comprised of polymers such as acrylates, rubbers, siloxanes, isobutylene, urethanes, olefins and similar materials. The interface layer should be suitably flexible, for example a PET polymer, a PET polymer that is strain-relieved with serpentine cut-outs, or an inherently flexible polymer such as a silicone rubber. The interface layer may be a mixture of these types of polymers, along with any necessary additives to obtain the desired properties, and may need to be prepared using initiators, curing agents, or surface preparation steps. In some embodiments, in addition to maintaining proper contact between the device and the wearer's skin, the interface layer 130 may also facilitate the flow of a sweat sample from the skin to the sensors 142, 144. In such embodiments, the interface layer should ideally wet and maintain moisture on the sensors 142, 144 during device use. In such embodiments, the interface layer 130 may be a thin sheet of agarose gel, or rayon, or may be a z-axis membrane, or other fluid porous membrane, that primarily allows fluid to flow perpendicularly to the skin surface. The interface layer may be secured to the device 120 with, for example, a double-sided medical tape backing of polyester, polyethylene, textiles, paper, PET, PEN, Kapton, polypropylene, PTFE, hook and loop fasteners, or other materials (not shown).

FIG. 1B depicts a top-down view of a disposable device component 120 featuring an alternate configuration. The depicted embodiment provides a number of advantages, including moving the device to a location within the headgear that subjects the device to less mechanical pressures than a location toward the center of the forehead. In particular, the device may be positioned to minimize changes to pressure against the sensors 142. In some embodiments, the sweat sensing device will include a wicking component 160 that is in fluid communication with the sweat sensors 142. The use of a wick 160 as disclosed will allow the device to be completely off the skin with the exception of the wick, and in some embodiments, one or more skin contact sensors. The wicking component 160 will be secured to the sensors 142 by an adhesive or other means that maintains consistent pressure of the wick against the sensors. The wicking component 160 will also be held with positive pressure against the skin 12 by the headgear mounting surface 105. In some embodiments, a spacing component (not shown) located between the mounting surface 105 and the wicking component 160 may be necessary to supply the required pressure against the skin. Such a spacing component may be, for example, a spring, a foam block, or a sponge. The wicking component 160 will have a non-porous backing, such as PET, to restrict the flow of sweat within the wick. The wicking material will preferably be of a material that does not absorb electrolytes in the sweat sample so that sweat electrolyte concentrations will not be altered during transport through the wick. Some embodiments will feature electromagnetic shielding materials (not shown) between the skin and the device 120. In some embodiments, the device will also include a wicking pump (not shown) that will be placed in fluid communication with the wicking component 160 downstream of the sensors. By absorbing or facilitating evaporation of the sample, the wicking pump will maintain a positive flow of sweat across the sensors 142, and move older sweat away from the sensors.

With further reference to FIGS. 1A and 1B, during use the headgear may move front to back or side to side, placing compressive, shearing and torsion forces on the interface layer 130 or wicking component 160. While the device remains securely fastened to the headgear mounting surface 105 via the adhesive layer, the interface layer or wicking component allows the device to move small amounts relative to the skin surface, facilitating wearer comfort and maintaining adequate contact with the skin to allow accurate sweat measurements. In this completely disposable configuration, the device may include onboard electronics, communication, processing, and power resources sufficient to enable the device to operate and communicate with the user. Alternately, these resources may be distributed in various ways between the device and the headgear.

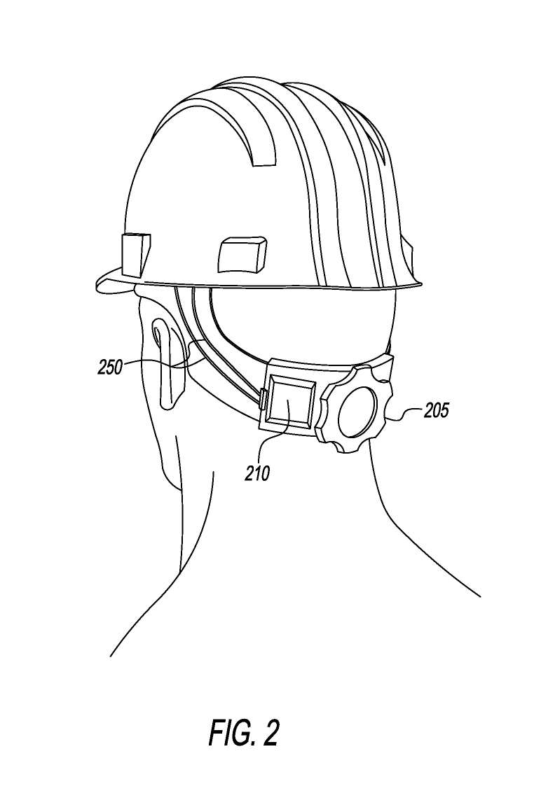

In other embodiments of the disclosed invention, the sweat sensing device may include a reusable component and a disposable component. As depicted in FIG. 2, the sweat sensing device includes a reusable component 210 that is integrated with a hardhat suspension system 205. Such suspensions may, for example, be modified aftermarket components that can be fitted into existing hardhats. The reusable component 210 is in electrical communication 250 with a disposable component (not shown). The device may rely completely on power, communications and processing resources that are located on the reusable component 210, or these resources may be distributed among the disposable and reusable components, and the headgear, in different combinations.

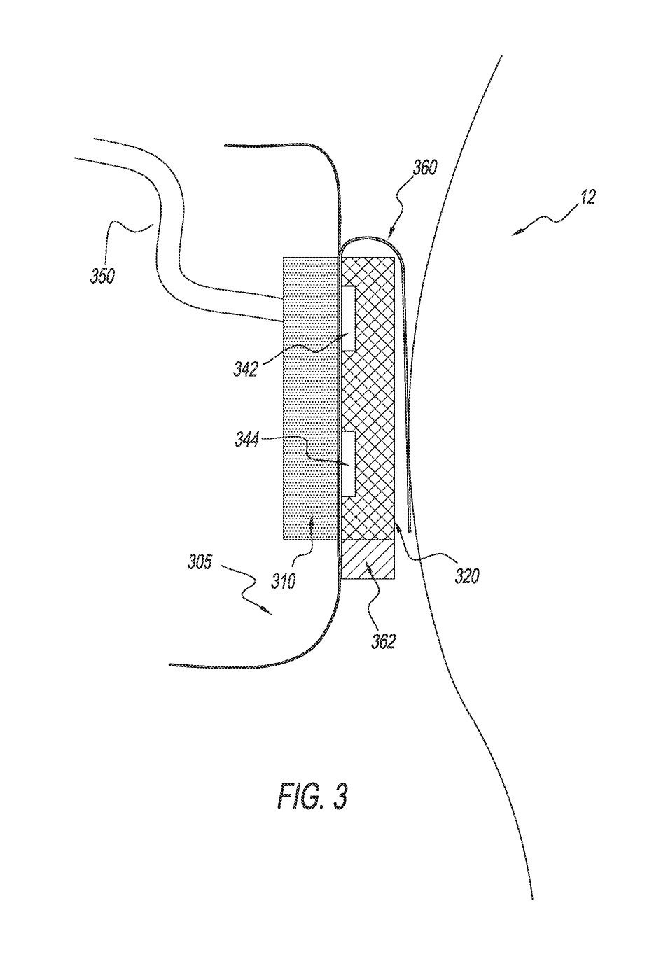

With reference to FIG. 3, the device has a reusable component 310 that provides secure connection with the headgear 305, and electrical connection 350 with headgear electronics, and includes, for example, onboard battery power, processing and communication capabilities (not shown). A disposable component 320 connects physically and electronically with the reusable component, and includes sweat sensors 342, 344, skin contact sensors (not shown), and basic electronics (not shown). Such physical connection may be through, for example, an adhesive, double-sided tape, clips, or hook and loop fasteners. A sweat wicking component 360 is in fluid communication with the sensors 342, 344, and should be secured to the sensors so that a consistent pressure is maintained between the sensors and the wick. During use, sweat will collect in the wick 360, and flow to the sensors 342, 344. The wicking component will have a partial sweat-impermeable backing to facilitate sweat flow across the sensors. In some embodiments, the wicking component will be in fluid communication with a wicking pump 362 that is placed downstream of the sensors, and which facilitates sweat flow across the sensors. The device is configured so that adequate and consistent positive pressure maintains the wicking component in adequate contact with the skin 12 to allow proper operation. Such pressure may be maintained by foam spacers, springs, sponges, clips, through the headgear itself, or other appropriate means, as long as adequate and consistent pressure is supplied that also is not great enough to cause delamination or other damage to the sensors. As described for the fully disposable device configuration, the individual components may support various combinations of electronics, processing capability, power supply, communications capability, etc., that are distributed among the disposable and reusable components, as well as the headgear.

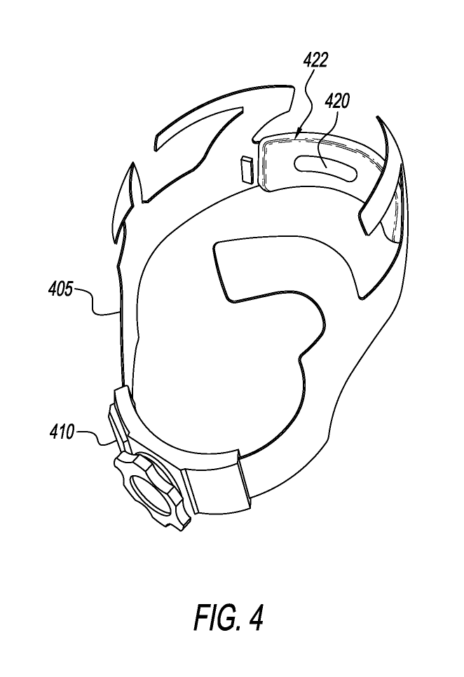

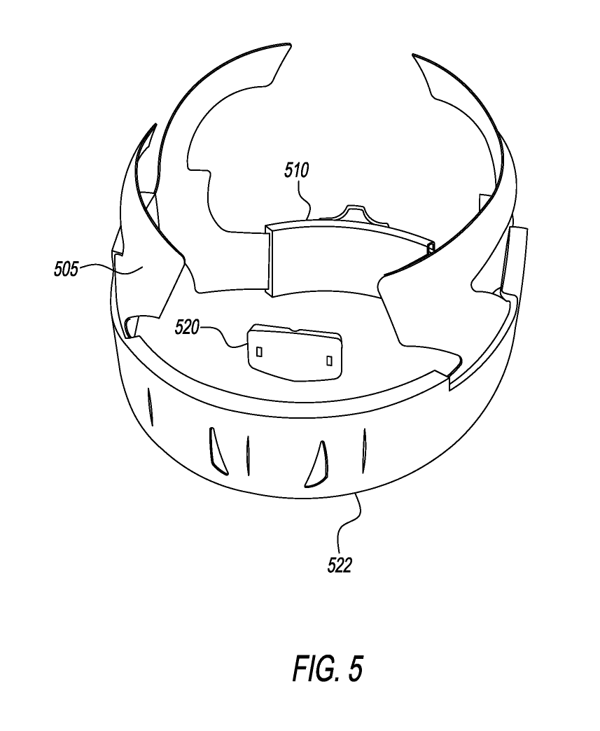

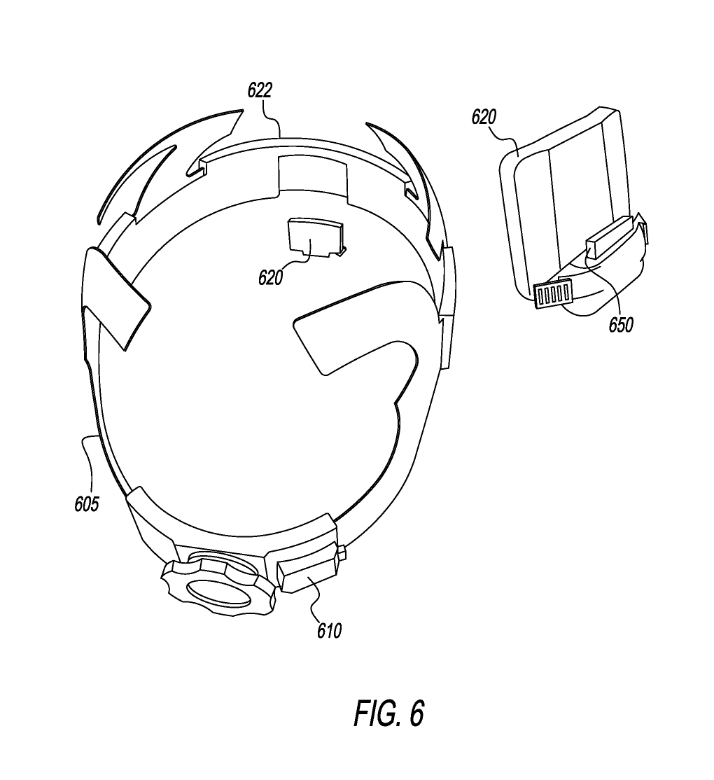

Several configurations of the disclosed invention are possible depending on the application needs of the device user. For example, with reference to FIG. 4, an embodiment of the disclosed invention is depicted in which the sweat sensing device includes a reusable component 410 that is integrated with the headgear 405, and is in electrical communication with a disposable component 420 that is integrated into the forehead pad 422, so that the normal function of the forehead pad is preserved. In such embodiments, the entire disposable component 420 and pad 422 may be replaceable, or the disposable component 420 could be independently replaceable. FIG. 5 depicts a view from the front of the headgear suspension 505, where an embodiment of the disclosed device is comprised of a reusable component 510 that is incorporated into the headgear suspension 505, and a disposable component 520 that clips into the headgear suspension 505 and is partially surrounded by the forehead pad 522. In another embodiment, as depicted in FIG. 6, the sweat sensing device includes a disposable cartridge 620 with electrical connector 650, which can be snapped into an electrical receptacle in the headgear suspension 605, so that the cartridge 620 is partially surrounded by the forehead pad 622. The device includes a reusable component 610 that is integrated into the suspension 605.

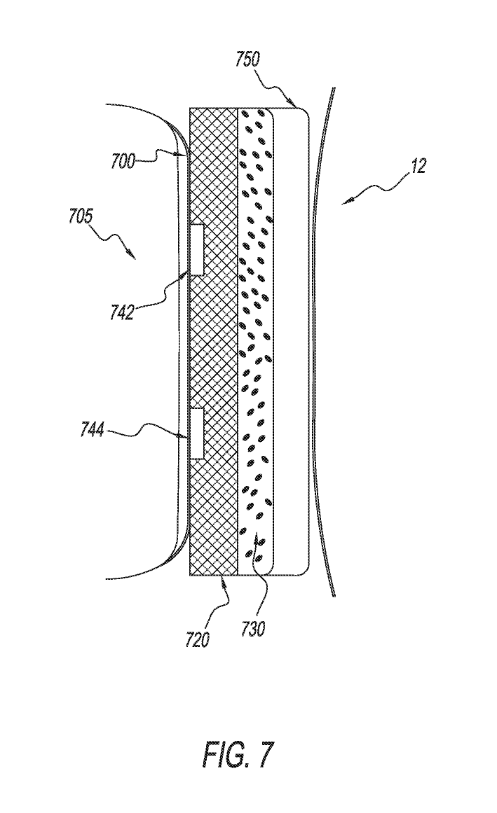

With reference to FIG. 7, in another embodiment of the disclosed invention, the device may include a protective material 750, such as a film or backing, over the device that protects the sweat sensors until needed to capture reliable data. The device includes a disposable component 720 that connects adhesively 700 and electronically with the headgear 705, or reusable component (not shown), and may include a flexible interface layer 730, sweat sensors 742, 744, a wicking component (not shown), skin contact sensors (not shown), and basic electronics (not shown). The protective material 750 may consist of a material that dissolves in the presence of sweat, or it may be peeled off prior to donning the headgear. The protective material would allow a sweat sensing device to be prepositioned in headgear, such as a firefighter helmet, without compromising the integrity of sweat sensor data during use. Prepositioning the sweat sensing device facilitates operational use by obviating the need for wearers to configure the device during short-notice response periods. Prepositioning also allows a power source for the device to be recharged, and may allow calibration, diagnostic, or other checks of the device prior to use.

The following examples are provided to help illustrate the present disclosure, and are not comprehensive or limiting in any manner.

### EXAMPLE 1

An advantage of the present disclosure would be the ease with which such a device may be incorporated into the operational activities of its wearers. For example, a firefighting company could use the devices to monitor the hydration level or cardiac stress of firefighters as they respond to an emergency call. At the start of a firefighter's shift, they are required to install a fresh sweat sensing device into the headband in their helmet suspension apparatus. During the shift, the helmet or device may be plugged in for recharging or for the performance of system diagnostics, for instance, to verify good electrical connections among the components. When a fire alarm is sounded, the firefighter places the helmet on her head, and the sweat sensing device is automatically positioned in contact with the firefighter's forehead. When the firefighter begins to sweat, a sweat dissolvable film protecting the device dissolves, and the device begins to take measurements.

### EXAMPLE 2

A military flight helmet is configured with an integrated partially disposable sweat sensing device in the forehead pad. During the pre-flight check of the equipment, an aircraft physiology technician inspects and readies the helmet's sweat sensing device for use. The device has a reusable component that is embedded in the exterior surface of the helmet, and which carries memory, processing and re-chargeable battery power. The technician performs an operational check of the reusable component and ensures it is in good electrical connection with the rest of the helmet's communication and sensing systems. Then, the technician clips a new disposable component into a receptacle in the forehead pad of the helmet. The disposable component includes sweat sensors for detecting K⁺, Na⁺, Cl⁻, pH, and cortisol, and has capacitive skin contact sensors, as well as electrical connections with the reusable portion of the device. Before donning the helmet, the fighter pilot removes the protective backing covering the disposable component. During the mission, the sweat sensing device performs periodic measurements to assess the pilot's hydration, stress and fatigue levels and communicates the results to the aircraft physiological monitoring system. The sweat sensing device continuously assesses the quality of skin contact and times its analyte readings accordingly. After the mission, the technician performs diagnostics on the reusable portion of the device, which is still operational and therefore does not need replacement, then the technician removes the used disposable component and plugs in the helmet to recharge the device battery.

This has been a description of the disclosed invention along with a preferred method of practicing the disclosed invention, however the invention itself should only be defined by the appended claims.

What is claimed is:

1. A sweat sensing device configured to be worn on an individual's skin and that is integrated into headgear, comprising:
one or more biofluid sensors for measuring a characteristic of an analyte in a biofluid sample;
an integration component to interface with the headgear;
a biofluid sample collector, wherein the collector is in fluidic contact with the one or more biofluid sensors; and
a skin contact sensor for measuring contact with the individual's skin.

2. The device of claim 1, wherein the device is at least partially reusable.

3. The device of claim 2, wherein the reusable component includes at least one of the following components: a power supply, a processing component, a memory component, and a communications component.

4. The device of claim 1, wherein the integration component interacts with a support structure of the headgear.

5. The device of claim 1, further comprising an electromagnetic shield, wherein the shield is configured to reduce electrical interference upon a measurement output of the one or more biofluid sensors.

6. The device of claim 1, wherein the biofluid collector further comprises a flexible interface to facilitate device contact with the skin.

7. The device of claim 6, where the flexible interface is comprised of one of the following materials: a PET polymer, a strain-relieved PET polymer, and silicone.

8. The device of claim 6, where the flexible interface is further configured to transport the biofluid sample.

9. The device of claim 8, where the flexible interface is comprised of one of the following materials: an agarose gel, a rayon sheet, a z-axis membrane, and a fluid porous membrane.

10. The device of claim 1, including a spacer to secure at least a portion of the device against the skin with a substantially consistent pressure level during device use.

11. The device of claim 10, wherein said pressure level is at least 265 pascals and no more than 75,000 pascals.

12. The device of claim 10, wherein the spacer is chosen from one of the following: a spring, a sponge, a set of clips, and a foam spacer.

13. The device of claim 1, wherein the biofluid sample collector comprises a microfluidic wick.

14. The device of claim 13, further comprising: a wicking pump that is in fluid communication with the microfluidic wick at a point downstream of the one or more biofluid sensors, and that is configured to transport the biofluid sample across said sensor.

15. A method of using the device of claim 1, comprising:
accessing a first alert condition that indicates the device is in adequate contact with the skin;
accessing a second alert condition that indicates the device is not in adequate contact with the skin;
determining whether the first alert condition or the second alert condition is satisfied based on a metric that includes a measurement by the skin contact sensor; and
facilitating a first operation state when the first alert condition is satisfied, and facilitating a second operation state when the second alert condition is satisfied.

16. The method of claim 15, further comprising tracking periods during which the device is in the first operation state, and periods during which the device is in the second operation state.

17. The method of claim 15, wherein the second operation state comprises one of the following: facilitating transmission of an alert communication to another device; and causing an alert to be locally presented.
